(19) United States
(12) Patent Application Publication
(10) Pub. No.: US 2024/0066153 A1
(43) Pub. Date: Feb. 29, 2024

(54) CEST-MRI Methods for Detection of Diseases

(71) Applicant: Mohammed Abdelraheem Albatany, London (CA)

(72) Inventor: Mohammed Abdelraheem Albatany, London (CA)

(21) Appl. No.: 17/176,123

(22) Filed: Feb. 15, 2021

(51) Int. Cl. A61K 49/14; A61K 49/18

(52) U.S. Cl. CPC A61K 49/14; A61K 49/1896

(57) ABSTRACT

This invention relates to the detection of the diseases by using Chemical Exchange Saturation Transfer (CEST)—Magnetic Resonance Imaging (MRI). Previously, there was no way to use CEST MRI to early detect and map the agents bind to amyloid beta protein, tau protein, alpha-synuclein protein in neurodegenerative diseases, and inflammation in many diseases such as neurodegenerative diseases. The exogenous agents can be used to produce MRI contrast, such as agents contain exchangeable protons such as hydroxyl, amine, and amide protons, and thereby provide imaging and mapping for detection of the amyloid beta protein, tau protein, alpha-synuclein protein, and aggregation proteins in neurodegenerative diseases and inflammation in many diseases such as neurodegenerative diseases, and other inflammatory diseases.

### CROSS-REFERENCE TO RELATED APPLICATION

This application claims the benefit of priority to U.S. Provisional Patent Application No. 62/979,461, filed Feb. 21, 2020, and U.S. Provisional Patent Application No. 63/015,187, filed Apr. 24, 2020, the entirety of which is incorporated by reference for any and all purposes.

### INTRODUCTION

Embodiments of the invention described in this specification generally relate to disease detection by imaging, and more particularly, to novel CEST-MRI methods for detection of diseases using chemical exchange saturation transfer (CEST) magnetic resonance imaging (MRI). Positron emission tomography (PET) scanners are used for detection and mapping amyloid beta plaques and neurofibrillary tangles in the brains of living people. PET scanners use radiotracers (tracers) and/or contrast agents to detect disease. When injected into the bloodstream of a patient, tracers or contrast agents cross quickly into the brain, where they bind to amyloid plaques or neurofibrillary tangles to mark them with emissions of mild radioactivity. Amyloid beta imaging is highly useful to enable people to begin therapy early enough in time to avoid or to significantly delay the development of neurodegenerative diseases.

In the progression of the disease, amyloid beta plaques precede tau tangles, and both cause eventual neural loss, the accumulation of amyloid in the brain has been identified as an early biomarker of some diseases, such as Alzheimer's disease. Also, MRI can be used to detect Alzheimer's disease but at a late stage where the structure of the brain had changed. The method presented in this invention can detect amyloid beta protein, tau protein, alpha-synuclein, and aggregation proteins in neurodegenerative diseases and other diseases by using novel CEST MRI methods before many years of the symptoms appear by using exogenous agents mostly safe for mammalians as a contrast.

Multiple sclerosis (MS) is a disabling disease of the brain and central nervous system. Magnetic resonance imaging (MRI) can diagnose MS and monitor disease progression by using T1-weighted brain MRI, but at a late stage, MRI cannot detect MS at an early stage. Some people with clinically definite MS do not initially show lesions on MRI at the time of diagnosis. Also, MRI uses a contrast agent or tracer to detect active inflammation; this scan will highlight the new lesions or lesions that are growing. Also, these MRI methods are unable to detect small lesions; MRI scans can detect damage in the central nervous system. In most cases, it helps to use a contrast agent, such as gadolinium, to better identify the lesions. However, gadolinium is expensive and has many side effects on humans, especially to humans with kidney diseases, also, cannot detect the lesions early. The method presented in this invention can detect multiple sclerosis (MS) by using novel CEST MRI before many years of the symptoms appear by using exogenous contrast such as flavonoids, polyphenol, NSAIDs and other drugs which have protons that can be saturated to induce CEST contrast such as amide, amine and hydroxide protons.

Method of imaging inflammation in Parkinson's disease (PD), epilepsy, ischemic stroke, and Alzheimer's disease (AD), Multiple sclerosis (MS), Huntington's disease (HD), Amyotrophic Lateral Sclerosis (ALS), concussion, traumatic brain injury, and other neurodegenerative diseases by administration of drugs or agents like Flavonoids such as luteolin, quercetin, rutin, taxifolin, resveratrol, myricetin, rhein, and others; congo red and it's analogs such as chrysamine-g; nordihydroguaiaretic acid; tannins from brown algae such as phlorotannins include eckol and it's derivatives and most polyphenols of seaweed; cannabinoids such as tetrahydrocannabinol (THC), cannabidiol (CBD), cannabigerol (CBG), and other cannabinoids; polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others and/or administration of compounds contain at least phenol group with exchangeable one proton or more and have ability to bind to human protein such as human serum albumin (HAS) which can be detected by CEST-MRI. These drugs can be accumulated in the inflammatory tissues and induce CEST contrast to detect the inflammation and accumulated at high expressed MCTs cells or tissue. Inflammation imaging by MRI can be used as early detection of disease, which associated with inflammation such as concussion, traumatic brain injury (TBI), AD, MS, HD, PK, ALS, and other inflammatory diseases.

Therefore, what is needed is a way to use MRI imaging to detect and map amyloid beta, tau protein, alpha-synuclein protein, and aggregation proteins in neurodegenerative diseases and other diseases, also, to detect and map inflammation in neurodegenerative diseases such as Parkinson's disease (PD), epilepsy, ischemic stroke, and Alzheimer's disease (AD), Multiple sclerosis (MS), Huntington's disease (HD), Amyotrophic Lateral Sclerosis (ALS), and other neurodegenerative diseases by using exogenous agents as contrast via MRI as opposed to PET scans which are longer in time, not as safe as CEST MRI, and provide lower resolution images than CEST MRI, and to do so without the need to inject expensive and unsafe contrast agents or tracers.

### DESCRIPTION

Novel CEST-MRI methods for the detection of diseases are disclosed. The novel CEST-MRI methods for detection of diseases such as amyloid beta, tau protein, alpha-synuclein, and aggregation proteins in neurodegenerative diseases by administration of drugs bind to amyloid beta and/or bind to tau protein and/or bind to alpha-synuclein and/or bind to aggregation proteins and induce contrast can be detected by CEST MRI. For example, agents like Flavonoids such as luteolin, quercetin, rutin, taxifolin, resveratrol, myricetin, rhein, and others; phenolic acid such as ferulic acid, caffeic acid, gallic acid, salicylic acid, and others; congo red and it's analogs such as chrysamine-g; nordihydroguaiaretic acid; tannins from brown algae such as phlorotannins include eckol and it's derivatives and most polyphenols of seaweed; cannabinoids such as tetrahydrocannabinol (THC), cannabidiol (CBD), cannabigerol (CBG), and other cannabinoids; polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others and/or administration of compounds contain at least phenol group with exchangeable one proton or more and have ability to bind to human protein such as human serum albumin (HAS) which can be detected by CEST-MRI.

In some embodiments, the novel CEST-MRI methods for detection of diseases includes a plurality of steps comprising (i) acquiring, by way of a magnetic resonance imaging (MRI) machine, a T2-image as an anatomical image, (ii) acquiring, by way of the MRI machine, a plurality of CEST images (before injection the agents) at saturation frequencies start from—20 ppm to 20 ppm with apart of 0.25 ppm, start and end saturation frequencies depend on agent's possible protons saturation frequencies that need to be administrated, for example quercetin can be start from −8 ppm to 8 ppm with apart higher than or less than of 0.25 ppm, therefore acquiring images for the brain before injection of quercetin from −8 ppm to 8 ppm with apart 0.25 ppm, (iii) acquiring, by way of the MRI machine, a plurality of CEST images (after injection the agents) at saturation frequencies start from—20 ppm to 20 ppm with apart higher than or less than of 0.25 ppm, start and end saturation frequencies depend on agent's possible saturation frequencies that need to be administrated, for example quercetin can be start from −8 ppm to 8 ppm with apart higher than or less than 0.25 ppm, therefore acquiring images for the brain after administration of quercetin from −8 ppm to 8 ppm with apart 0.25 ppm, using the saturation power and duration depend on the agents need to be administrated, if the agents contain exchangeable (hydroxyl protons and/or amine protons), these agents can be saturated by high power and short duration such as using 4.9 μT or less and 1 sec saturation time. Agents contain amid protons need to be saturated by lower power and short duration, such as using 1.5 μT and 4 sec saturation time.

(iv) (MTR) asymmetry before administration of the agents, acquiring CEST images at specific chemical shifts and at frequencies that decrease the magnetization of the exchangeable protons (Δω) of the administrated agents and also acquiring CEST images at specific chemical shifts at the opposite side of the same frequency (−Δω) which is decrease the magnetization of the exchangeable protons of the administrated agents at frequency (−Δω) according to MTR_{asym-pre} (Δω)=Sₛₐₜ (−Δω)/Sₒ−Sₛₐₜ (Δω)/Sₒ, where Sₛₐₜ and Sₒ are the signal intensity obtained with and without selective saturation, respectively, for example, for quercetin administration acquire images for the brain of mammalian at 5 ppm and −5 ppm before administration of quercetin.

(v) (MTR) asymmetry after administration of the agents, such as administration of the agents by injections or orally to the animals or humans (mammalians), acquiring CEST images at specific chemical shifts and at frequencies that decrease the magnetization of the exchangeable protons (AO) of the administrated agents and also acquiring CEST images at specific chemical shifts at the opposite side of the same frequency (−Δω) which is decrease the magnetization of the exchangeable protons of the administrated agents at frequency (−Δω) according to MTR_{asym-post} (Δω)=Sₛₐₜ (−Δω)/Sₒ−Sₛₐₜ (Δω)/Sₒ, where Sₛₐₜ and Sₒ are the signal intensity obtained with and without selective saturation, respectively, for example, for quercetin administration acquire images for the brain of mammalian at 5 ppm and −5 ppm after administration of quercetin.

(vi) calculating the difference in MTR_{asym} as MTR_{asym-contrast} (Δω)=MTR_{asym-post} (Δω)−MTR_{asym-pre} (Δω).
(vii) detecting disease and mapping disease severity based on the calculated MTR_{asym-contrast} (Δω), as the MTR_{asym-contrast} (Δω) increase as the disease is increased in severity.

In some embodiments of this invention, (Δω) of the administrated agents can be calculated from phantoms mixed these agents with solution contain water and measure the (AO) that have best peaks in the phantoms by MTR_{asym} spectrum (MTR_{asym} against frequencies (Δω)).

Method of imaging and detection of amyloid beta protein, and/or tau protein and/or alpha-synuclein protein, and/or aggregation proteins by agents, drugs, natural compounds, and any other chemical compounds bind to amyloid beta protein, and/or tau protein and/or alpha-synuclein protein and/or aggregation proteins by using CEST-MRI for agents contain exchangeable protons such as hydroxyl, amine, and amide protons, and thereby provide imaging and mapping for detection of the amyloid beta protein, tau protein, alpha-synuclein protein, and aggregation proteins in neurodegenerative diseases and other diseases.

Also, method of imaging and detection of inflammation by agents, drugs, natural compounds, and any other chemical compounds bind to and accumulated in the inflammatory tissues by using CEST-MRI for agents contain exchangeable protons such as hydroxyl, amine, and amide protons, and thereby provide imaging and mapping for detection of the inflammation in many diseases such as neurodegenerative diseases, and other inflammatory diseases except cancer.

There are many agents can bind to amyloid beta and/or tau protein and/or alpha-synuclein and/or aggregation proteins and can be detected by CEST-MRI and induce contrast after 10 mins to 3 hrs of administration these agents orally or by injections to animals or humans, also, it is possible to enhance the CEST contrast by giving these agents as a daily treatment for few days to enhance the binding of these agents to the target amyloid beta and/or tau protein and/or alpha-synuclein and/or aggregation proteins for better detection and to enhance the CEST contrast compare to baseline images before the administration of these agents. For example, administration of agents such as Flavonoids such as luteolin, quercetin, rutin, taxifolin, resveratrol, myricetin, rhein, and others; phenolic acid such as ferulic acid, caffeic acid, gallic acid, salicylic acid, and others; congo red and it's analogs such as chrysamine-g; nordihydroguaiaretic acid; tannins from brown algae such as phlorotannins include eckol and it's derivatives and most polyphenols of seaweed; cannabinoids such as tetrahydrocannabinol (THC), cannabidiol (CBD), cannabigerol (CBG), and other cannabinoids; polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others and/or administration of compounds contain at least phenol group with exchangeable one proton or more and have ability to bind to human protein such as human serum albumin (HAS) which can be detected by CEST-MRI.

Method of imaging inflammation in concussion, traumatic brain injury, AD, MS, PD, HD, ALS, and other neurodegenerative diseases by administration of drugs. For example, agents such as Flavonoids such as luteolin, quercetin, rutin, taxifolin, resveratrol, myricetin, rhein, and others; congo red and it's analogs such as chrysamine-g; nordihydroguaiaretic acid; tannins from brown algae such as phlorotannins include eckol and it's derivatives and most polyphenols of seaweed; cannabinoids such as tetrahydrocannabinol (THC), cannabidiol (CBD), cannabigerol (CBG), and other cannabinoids; polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others).

These drugs can be accumulated in the inflammation tissue and induce CEST contrast to detect the inflammation and high expressed MCTs cells or tissue. Inflammation imaging by MRI can be used as early detection of disease, which associated with inflammation such as concussion, traumatic brain injury, AD, MS, HD, PK, ALS, and other inflammatory diseases.
Novel CEST-MRI methods for the detection of diseases using CEST MRI of the present disclosure may produce or result in the following elements. This list of possible constituent elements is intended to be exemplary only, and it is not intended that this list be used to limit novel CEST-MRI methods for the detection of diseases using CEST MRI of the present application to just these elements. Persons having ordinary skill in the art relevant to the present disclosure may understand there to be equivalent elements that may be substituted within the present disclosure without changing the essential function or operation of the novel CEST-MRI methods for detection of diseases using CEST MRI.
Step 1. Acquiring anatomical T₂-image(s) of specific areas of the brain, which are the areas of focus as expected under the effects of certain diseases. For instance, specific areas of the brain include the hippocampus and the cortex in Alzheimer's disease
Step 2. CEST images (before administration of the agents)
Step 3. CEST images (after administration of the agents)
Step 4. Calculate (MTR) asymmetry before administration of the agents as MTR_{asym-pre}.
Step 5. Calculate (MTR) asymmetry after administration of the agents as MTR_{asym-post}
Step 6. Calculating the difference in MTR_{asym} as MTR_{asym-contrast} (Δω)=MTR_{asym-post} (Δω)−MTR_{asym-pre} (Δω).
Step 7. Detecting disease and mapping disease severity based on the calculated MTR_{asym-contrast} (Δω), as the MTR_{asym-contrast} (Δω) increase as the disease is increased in severity.
Novel CEST-MRI methods for detection of disease mapping using CEST MRI of the present disclosure support most of the pulse sequences for CEST images. For example, one may use fast spin-echo (FSE) pulse sequence by using the following parameters: TR=3.2 s; TE=6.5 ms; FOV=212×190 mm²; matrix size=256×256; slice thickness=4.2 mm; turbo-spin-echo factor=40; and single slice acquisition. The RF saturation section includes a series of four block RF saturation pulses (100 ms duration each and 4 μT amplitude) at 3 Tesla for the human, the optimization of the saturation power and duration depend on the imaging target such as using exogenous contrast; some agents need higher power and short duration especially that contain amine and hydroxide protons.
To use Novel CEST-MRI methods for detection of diseases using CEST MRI of the present disclosure, a person working on MRI (an MRI operator) can follow the general steps noted above (Step 1, Step 2, Step 3, Step 4, Step 5, Step 6, and Step 7). Thus, to obtain accurate disease detection and severity mapped results, the MRI operator drives the actions noted in the steps of the methods by control operations with respect to the CEST MRI machine, which captures the images, and by interaction with a computing device, which performs computations, thereby allowing the MRI operator to follow the general steps (Step 1, Step 2, Step 3, Step 4, and Step 5, Step 6, and Step 7).

The MRI operator may carry out more specific steps. Furthermore, the MRI operator can produce MTR_{asym-pre}, MTR_{asym-post}, and MTR_{asym-contrast} by using MATLAB (MathWorks) on a computing device and thereby make determinations of diseases that may be present.

MTR_{asym-post} map and MTR_{asym-contrast} map can be used to detect the agents that bind to amyloid beta and/or tau protein and/or alpha-synuclein protein and/or aggregation proteins in neurodegenerative diseases and other diseases, MTR_{asym-post} will be increased after administration of agents compared to MTR_{asym-pre}, which refer to increase binding of the agents to these proteins (amyloid beta, tau, alpha-synuclein, aggregation proteins) as the disease is severe, the MTR_{asym-post} is significantly increased as amyloid beta and/or tau protein and/or alpha-synuclein protein and/or aggregation proteins increased in neurodegenerative diseases and other diseases. Also, MTR_{asym-contrast} map can be used as difference map to the detection of the agents bind to amyloid beta and/or tau protein and/or alpha-synuclein protein aggregation proteins in neurodegenerative diseases and other diseases as MTR_{asym-contrast} is increased significantly as amyloid beta and/or tau protein and/or alpha-synuclein protein and/or aggregation proteins increased in neurodegenerative diseases and other diseases.

MTR_{asym-post} map and MTR_{asym-contrast} map can be used to detect the agents that bind to inflammatory tissues in neurodegenerative diseases, and other inflammatory diseases. MTR_{asym-post} will be increased after administration of agents compared to MTR_{asym-pre}, which refer to increase binding of the agents to inflammation tissues in neurodegenerative diseases, and other inflammatory diseases as the inflammation are severe, the MTR_{asym-post} is significantly as increased in inflammation tissues in neurodegenerative diseases, and other inflammatory diseases. Also, MTR_{asym-contrast} map can be used as difference map to the detection of agents bind to inflammation tissues in neurodegenerative diseases, and other inflammatory diseases which can be detected by CEST-MRI and induce contrast after 10 mins to 3 hrs of administration these agents orally or by injections to animals or humans, MTR_{asym-contrast} is increased significantly as the inflammation is increased.

1. Novel CEST-MRI methods for detection of diseases comprising: imaging of agents bind to amyloid beta proteins and/or tau proteins by decrease magnetization of the protons of these agents and transfer of these magnetizations to bulk water and surrounding tissues by way of a magnetic resonance imaging (MRI) machine, acquiring, by way of MRI machine, a T2-image as an anatomical image; acquiring, by way of the MRI machine, a plurality of CEST images (before injection the agents); acquiring, by way of the MRI machine, a plurality of CEST images (after administration of the agents); Calculate (MTR) asymmetry before administration of the agents MTRasym-pre; and Calculate (MTR) asymmetry after administration of the agents as MTRasym-post; and calculating the difference in MTRasym as
MTRasym-contrast (Δω)=MTRasym-post (Δω)−MTRasym-pre (Δω); detecting the agents (to enhance the CEST detection, it is possible to administration orally or by injections more than one agent) that can bind to amyloid beta proteins and/or tau proteins based on the calculated MTRasym-contrast (Aw), as the MTRasym-contrast (Δω) increase as the binding agents to the target amyloid proteins and/or tau proteins increase.

2. Novel CEST-MRI methods for detection of diseases comprising: imaging of agents bind to alpha-synuclein proteins and/or protein aggregation by decrease magnetization of the protons of these agents and transfer of these magnetizations to bulk water and surrounding tissues by way of magnetic resonance imaging (MRI) machine, acquiring, by way of MRI machine, a T2-image as an anatomical image; acquiring, by way of the MRI machine, a plurality of CEST images (before injection the agents); acquiring, by way of the MRI machine, a plurality of CEST images (after administration of the agents); Calculate (MTR) asymmetry before administration of the agents MTRasym-pre; and Calculate (MTR) asymmetry after administration of the agents as MTRasym-post; and calculating the difference in MTRasym as
MTRasym-contrast (Δω)=MTRasym-post (Δω)−MTRasym-pre (Δω); detecting the agents (to enhance the CEST detection, it is possible to administration orally or by injection more than one agent) that can bind to alpha-synuclein proteins and/or protein aggregation based on the calculated MTRasym-contrast (Δω), as the MTRasym-contrast (Δω) increase as the binding agents to the target alpha-synuclein proteins and/or protein aggregation increase.

3. Novel CEST-MRI methods for detection of diseases of claim 1, comprising: administration of agents binds to amyloid beta proteins and tau proteins orally, or by injection or administration multiple agents target and bind to amyloid beta protein and tau proteins separately to enhance the detection of amyloid beta protein and tau protein in the same time which can be detected by CEST-MRI.

4. Novel CEST-MRI methods for detection of diseases claim 1, comprising: administration of agents binds to amyloid beta proteins and alpha-synuclein proteins orally, or by injection or administration multiple agents target and bind to amyloid beta proteins and alpha-synuclein proteins separately to enhance the detection of amyloid beta protein and alpha-synuclein proteins in the same time which can be detected by CEST-MRI.

5. Novel CEST-MRI methods for detection of diseases of claim 1, comprising: administration of agents binds to amyloid beta, tau, and alpha-synuclein proteins orally or by injection or administration multiple agents target and bind to amyloid beta, tau, and alpha-synuclein proteins separately to enhance the detection of amyloid beta, tau, and alpha-synuclein proteins in the same time which can be detected by CEST-MRI.

6. Novel CEST-MRI methods for detection of diseases of claim 1, comprising: administration of agents binds to tau proteins and alpha-synuclein proteins orally, or by injection or administration multiple agents target and bind to tau proteins and alpha-synuclein proteins separately to enhance the detection of tau proteins and alpha-synuclein proteins in the same time which can be detected by CEST-MRI.

7. Novel CEST-MRI methods for detection of diseases of claim 2, comprising: administration of agents bind to the protein aggregation orally, or by injection or administration multiple agents target and bind to the protein aggregation separately to enhance the detection of the protein aggregation in the same time which can be detected by CEST-MRI.

8. Novel-CEST-MRI methods for detection of diseases of claim 1, comprising: administration of agents binds to amyloid beta and/or tau proteins orally or by injection by using flavonoids such as luteolin, quercetin, rutin, taxifolin, resveratrol, myricetin; rhein, and others; tannins from brown algae such as phlorotannins include eckol and it's derivatives and most polyphenols of seaweed; phenolic acid such as ferulic acid, caffeic acid, gallic acid, salicylic acid, and others; congo red and it's analogs such as chrysamine-g nordihydroguaiaretic acid; cannabinoids such as tetrahydrocannabinol (THC), cannabidiol (CBD), cannabigerol (CBG), and other cannabinoids; polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others.

9. Novel CEST-MRI methods for detection of diseases of claim 2, comprising: administration of agents bind to alpha-synuclein proteins and/or protein aggregation orally or by injection by using flavonoids such as luteolin, quercetin, rutin, taxifolin, resveratrol, myricetin, rhein, and others; tannins from brown algae such as phlorotannins include eckol and it's derivatives and most polyphenols of seaweed; phenolic acid such as ferulic acid, caffeic acid, gallic acid, salicylic acid, and others; congo red and it's analogues such as chrysamine-g; nordihydroguaiaretic acid; cannabinoids such as tetrahydrocannabinol (THC), cannabidiol (CBD), cannabigerol (CBG), and other cannabinoids; polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocarechin, and epigallocatechin gallate, and others.

10. Novel CEST-MRI methods for detection of diseases comprising: imaging of agents bind to inflammatory tissues and immune cells except in cancer by decrease magnetization of the protons of these agents and transfer of these magnetizations to bulk water and surrounding tissues by way of a magnetic resonance imaging (MRI) machine, acquiring, by way of MRI machine, a T2-image as an anatomical image; acquiring, by way of the MRI machine, a plurality of CEST images (before injection the agents); acquiring, by way of the MRI machine, a plurality of CEST images (after administration of the agents); Calculate (MTR) asymmetry before administration of the agents MTRasym-pre; and Calculate (MTR) asymmetry after administration of the agents as MTRasym-post; and calculating the difference in MTRasym as
MTRasym-contrast (Δω)=MTRasym-post (Δω)−MTRasym-pre (Δω); detecting the accumulation of the agents (to enhance the CEST detection, it is possible to administration orally or by injection more than one agent) that can bind to inflammatory tissues and immune cells based on the calculated MTRasym-contrast (Δω), as the MTRasym-contrast (Δω) increase as the agents accumulated in the inflammatory tissues.

11. Novel CEST-MRI methods for detection of diseases of claim 10, comprising: administration of agents bind and accumulate in the inflammatory tissues and immune cells except in cancer by using flavonoids such as luteolin, quercetin, rutin, taxifolin, resveratrol, myricetin, rhein, and others; congo red and it's analogs such as chrysamine-g; nordihydmguaiaretic acid; cannabinoids such as tetrahydrocannabinol (THC), cannabidiol (CBD), cannabigerol (CBG), and other cannabinoids; tannins from brown algae such as phlorotannins include eckol and it's derivatives and most polyphenols of seaweed; polyphenols such as curcumin; rosmarinic acid; phenylindane, silibinin, silymarin, thearubigins, theaflavin, and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others.

12. Novel CEST-MRI methods for detection of diseases of claim 1, wherein detecting disease and mapping disease severity for mammalians comprises amyloid beta diseases (amyloidosis) such as Alzheimer's disease, Parkinson disease, Amyotrophic lateral sclerosis, Huntington's disease, type H diabetes, epilepsy, Down syndrome and other neurodegenerative diseases caused by an accumulation of amyloid beta proteins.

13. Novel CEST-MRI methods for detection of diseases of claim 1, wherein detecting disease and mapping disease severity for mammalians comprises tau proteins diseases (tauopathy) such as Alzheimer's disease, Parkinson disease, Huntington's disease, type II diabetes, Down syndrome and other neurodegenerative diseases caused by an accumulation of tau proteins.

14. Novel CEST-MRI methods for detection of diseases of claim 2, wherein detecting disease and mapping disease severity for mammalians comprises alpha synuclein proteins diseases (synucleinopathathies) such as Alzheimer's disease, Parkinson disease, Huntington's disease, type II diabetes, Down syndrome and other neurodegenerative diseases caused by an accumulation of alpha synuclein proteins and Lewy bodies.

15. Novel CEST-MRI methods for detection of diseases of claim 2, wherein detecting disease and mapping disease severity for mammalians comprises the protein aggregation diseases (Protein aggregation diseases (PAD)) such as all fibrosis diseases include pulmonary fibrosis and liver fibrosis, Alzheimer's disease, Parkinson disease, Huntington's disease, type II diabetes, epilepsy, Down-syndrome and other neurodegenerative diseases caused by an accumulation of protein aggregation.

16. Novel CEST-MRI methods for detection of diseases of claim 1, comprising: administration of agents binds to amyloid beta and/or tau proteins orally or by injection by using administration of compounds contain at least phenol group with exchangeable one proton or more and have ability to bind to human protein such as human serum albumin (HAS) and/or bind to amyloid beta and/or tau proteins which can be detected by CEST-MRI.

17. Novel CEST-MRI methods for detection of diseases of claim 2, comprising: administration of agents bind to alpha-synuclein proteins and/or protein aggregation orally or by injection by administration of compounds contain at least phenol group with exchangeable one proton or more and have ability to bind to human protein such as human serum albumin (HAS) and/or bind to alpha-synuclein proteins and/or protein aggregation which can be detected by CEST-MRI.

18. Novel CEST-MRI methods for detection of diseases of claim 10, wherein detecting disease and mapping disease severity for mammalians comprises the inflammation at any part of the body and also, the inflammatory diseases such as diseases of the skin, diseases of the nervous system, immune diseases, diseases of the musculoskeletal system, concussion, traumatic brain injury, Alzheimer's disease, Parkinson disease, Huntington's disease, epilepsy, Down syndrome, and other neurodegenerative diseases.

19. Novel CEST-MRI methods for detection of diseases of claim 10, comprising: administration of agents bind and accumulate in the inflammatory tissues and immune cells except in cancer by administration of compounds contain at least phenol group with exchangeable one proton or more and have ability to bind to human protein such as human serum albumin (HAS) and accumulate in the inflammatory tissues which can be detected by CEST-MRI.

20. Novel CEST-MRI methods for detection of diseases of claim 10, comprising: administration of agents bind and accumulate in the inflammatory tissues, amyloid beta, tau proteins, alpha-synuclein proteins, protein aggregation and immune cells by administration of compounds contain at least phenol group with exchangeable one proton or more and have ability to bind to human protein such as human serum albumin (HAS) and accumulate in the inflammatory tissues and bind to amyloid beta, tau proteins, alpha-synuclein proteins, protein aggregation and immune cells which can be detected by CEST-MRI.
